(19) United States
(12) Patent Application Publication
(10) Pub. No.: US 2011/0053898 A1
(43) Pub. Date: Mar. 3, 2011

(54) TOPICAL COMPOSITION COMPRISING VITAMIN D ANALOGUE AND CORTICOSTEROIDS

(75) Inventors: Kamal Mehta, Bhilwara (IN); Amol Mandhare, Thane (West) (IN); Shantesh Patil, Pune (IN); Tapan Nayak, Cuttack (IN); Lalatendu Panigrahi, Bhubaneswar (IN)

(73) Assignee: GLENMARK GENERICS LTD, Mumbai (IN)

(21) Appl. No.: 12/862,043

(22) Filed: Aug. 24, 2010

(30) Foreign Application Priority Data
Aug. 26, 2009 (IN) 1961/MUM/2009
Jun. 18, 2010 (IN) 1826/MUM/2010

(51) Int. Cl. A61K 31/59; A61P 17/06

(52) U.S. Cl. 514/167

(57) ABSTRACT

A topical pharmaceutical composition comprising therapeutically effective amounts of vitamin D or vitamin D analogues, corticosteroid and a straight chain C₆-C₂₈ fatty alcohol.

### PRIORITY

This application claims priority to Indian Provisional Applications No. 1961/MUM/2009, filed on Aug. 26, 2009 and 1826/MUM/2010 filed on Jun. 18, 2010, the contents of which are hereby incorporated by reference.

### BACKGROUND OF THE INVENTION

1. Technical Field

The present invention deals with pharmaceutical compositions for dermal use that contain vitamin D or vitamin D analogue and at least one corticosteroid and a process for their preparation.

2. Description of the Related Art

Calcipotriene, represented below as the chemical structure I, was first described in International patent application WO 87/00834.

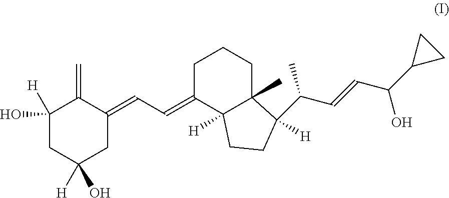

Calcipotriene is a vitamin-D₃ derivative, about 1% as powerful as the natural hormone calcitriol. Calcipotriene controls the rapid growth of skin cells. Calcipotriene is used to treat psoriasis, wherein it works by controlling the overproduction of skin cells in areas affected by psoriasis.

Betamethasone dipropionate (9-Fluoro-11.beta.,17,21-trihydroxy-16.beta.-methylpregna-1,4-diene-3,20-dione 17,21-dipropionate) is a topical corticosteroid. It has anti-inflammatory, antipruritic, vasoconstrictive and immunosuppressive properties. The structure of betamethasone dipropionate is shown below:

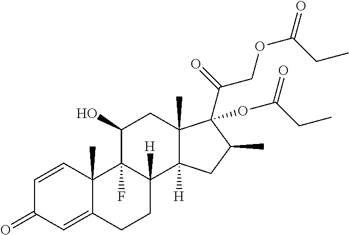

Topical steroid compounds, like corticosteroids, and vitamin D or vitamin D analogues, like calcipotriene (calcipotriol) or calcitriol, are used in combination to treat psoriasis or other inflammatory diseases. Taclonex® ointment contains two active ingredients, betamethasone dipropionate and calcipotriol hydrate.

### SUMMARY OF THE INVENTION

The present invention provides, a stable pharmaceutical composition, comprising a therapeutically effective amount of vitamin D or vitamin D analogues and corticosteroid.

The present invention provides a stable pharmaceutical composition, preferably a semisolid dosage form, comprising therapeutically effective amounts of vitamin D or vitamin D analogues, corticosteroid and fatty alcohol.

The present invention provides a topical pharmaceutical composition comprising therapeutically effective amounts of vitamin D or vitamin D analogues, corticosteroid and a straight chain C₆-C₂₈ fatty alcohol.

The present invention provides a stable topical pharmaceutical composition comprising a therapeutically effective amounts of calcipotriene, corticosteroid and straight chain C₆-C₂₈ fatty alcohol.

The present invention provides a stable ointment comprising therapeutically effective amounts of anhydrous calcipotriene, corticosteroid and oleyl alcohol.

The present invention provides a stable ointment comprising therapeutically effective amounts of anhydrous calcipotriene, corticosteroid and myristyl alcohol.

The present invention provides a process for the preparation of a stable pharmaceutical composition in a semisolid dosage form comprising: (a) dissolving calcipotriene and corticosteroid in fatty alcohol; (b) adding this to semisolid base comprising white petrolatum and mineral oil at about 65° C. to about 70° C. with stifling.

The present invention provides a process for the preparation of a stable ointment comprising: (a) dissolving calcipotriene and corticosteroid in fatty alcohol; (b) adding this to ointment base comprising white petrolatum and mineral oil at about 65° C. to about 70° C. with stirring.

### DETAILED DESCRIPTION OF THE PREFERRED EMBODIMENTS

The present invention provides a stable topical pharmaceutical composition, comprising anhydrous calcipotriene and corticosteroids dissolved in straight chain fatty alcohols.

Since a corticosteroid, like betamethasone dipropionate and a vitamin analogue, like calcipotriol hydrate require different pH environments for optimum stability, there are limited sources of references available that may teach pharmaceutical compositions comprising vitamin D or vitamin D analogues and corticosteroids. Illustratively, U.S. Pat. No. 6,753,013, which is included herein as reference in its entirety, describes a topical pharmaceutical composition, comprising a combination of a vitamin D analogue and a corticosteroid.

U.S. Patent Application 2008/0064669 (the '669 application) describes pharmaceutical compositions comprising vitamin D analogues (e.g. calcipotriene) and at least one corticosteroid compound (e.g. betamethasone dipropionate). The '669 application describes the preparations of the compositions by using a solvent selected from the group consisting of triglycerides, sorbitan, sorbitan fatty esters, cetearyl glucoside, PEG-n sorbitan stearate, acrylamide/sodium acryloyldimethyl taurate copolymers, and mixtures thereof.

The art still requires and seeks to answer the complexities in developing stable topical compositions comprising vitamin D or vitamin D analogues and corticosteroids, due to the different pH requirements of each active principle, where such a stable pharmaceutical composition comprising vitamin D or vitamin D analogues and corticosteroids, and/or a process for their preparation, is highly desired.

The present invention provides a pharmaceutical stable ointment comprising a therapeutically effective amount of anhydrous calcipotriene and corticosteroid dissolved in a fatty alcohol wherein the fatty alcohol is a straight chain fatty alcohols having a carbon chain length of C₆-C₂₈, preferably of C₈-C₂₈, more preferably of C₁₂-C₂₄.

The present invention provides a stable ointment comprising a therapeutically effective amount of anhydrous calcipotriene and corticosteroid dissolved in about 5% to about 15% w/w, preferably about 8% to about 10% w/w oleyl alcohol (Super Refined® Novol NF available from CRODA USA) having straight chain of 18 carbon atoms.

The present invention provides a stable ointment comprising a therapeutically effective amount of anhydrous calcipotriene and corticosteroid dissolved in about 1% to about 10% w/w, preferably about 2% to about 6% w/w myristyl alcohol (Speziol®C14 Pharma, available from Cognis, Germany) having straight chain of 14 carbon atoms.

Preferably, the present invention provides an ointment base selected from the group comprising mineral oil (Drakeol® 35, available from Penreco), white petrolatum (Ultima®, available from Penreco) and optionally an antioxidant such as butylated hydroxytoluene (BHT).

Topical compositions, usually, have a semisolid consistency, and are intended to be applied over the affected area, such as the skin or scalp, for therapeutic or protective action. The present invention provides topical pharmaceutical composition, which includes ointment, cream, lotion, scalp lotion, liniment or other spreadable liquid or semi liquid preparation, which is, preferably, non-aqueous or in the form of an oil-in-water or water-in-oil emulsion. Preferably, the composition of the invention is in the form of an ointment.

The present invention uses excipients selected from the category which are physicochemically compatible with the anhydrous form of calcipotriene and with corticosteroids in a semisolid dosage form.

The term or terms “vitamin D or vitamin D analogue”, as used herein, of the present invention can be selected from the group consisting of seocalcitol, calcipotriol, calcitriol, tacalcitol, maxacalcitol, paricalcitol, falecalcitriol, 1.alpha.,24S-dihydroxy-vitamin D2, 1(S),3(R)-dihydroxy-20(R)-[((3-(2-hydroxy-2-propyl)-phenyl)-methoxy)-methyl]-9,10-seco-pregna-5(Z),7(E),10(19)-triene and mixtures thereof.

The term, “calcipotriene”, as used herein, refers to anhydrous calcipotriene or hydrate forms of calcipotriene; however the anhydrous calcipotriene is the preferred form for the purpose of the present invention.

The corticosteroid compounds include betamethasone (9-fluoro-11,17,21-trihydroxy-16-methylpregna-1,4-diene-3,20-dione) and esters thereof such as the 21-acetate, 17-adamantoate, 17-benzoate, 17-valerate, and 17,21-dipropionate; alclomethasone and esters thereof such as the dipropionate; clobetasole and esters thereof such as the propionate; clobetasone and esters thereof such as the 17-butyrate; desoximetasone; diflucortolone and esters thereof, diflorasone and esters thereof such as the diacetate; fluocinonide; flumetasone and esters thereof such as the pivalate; fluocinolone and ethers and esters thereof such as the acetonide; fluticasone and esters thereof such as the propionate; fluprednidene and esters thereof such as the acetate; halcinonide; hydrocortisone and esters thereof such as the -17-butyrate; mometasone and esters thereof such as the furoate; and triamcinolone and ethers and esters thereof such as the acetonide. Betamethasone or esters thereof such as the valerate or dipropionate are preferred.

The topical composition of the present invention comprises at least one solvent and semisolid base. The semisolid base of the present invention may comprise one or more ingredients, selected from the group comprising of, but not limited to, petrolatum or white petrolatum or paraffins, mineral oil, cetostearyl alcohol, cetyl alcohol, lanolin, stearic acid, crosslinked acrylic acid polymers such as the “carbomer” family of polymers, e.g., carboxypolyalkylenes that may be obtained commercially under the Carbopol® such as Carbopol®940, polyethylene oxides, polyoxyethylene-polyoxypropylene copolymers, polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin or mixtures thereof. The semisolid base may further comprise co-solvents, emulsifiers, anti-oxidants, preservatives, chelating agent, buffering agent and the like.

The solvents of the present invention, which are used to dissolve vitamin D or vitamin D analogue (e.g. anhydrous calcipotriene) and corticosteroids (e.g. betamethasone dipropionate) mainly comprise fatty alcohols, derived from natural fats and oils, which are high molecular weight straight chain fatty alcohols with a carbon chain length of C₆-C₂₈. This would include capryl (C8), pelargonic (C9), capric (C10), lauryl (C12), myristyl (C14), cetyl (or palmityl (C16), stearyl (C18), oleyl (C18, unsaturated), and linoleyl (C18, polyunsaturated), arachidyl (C20) alcohols. For the purpose of present invention, super refined oleyl alcohol (Super Refined® Novol NF available from CRODA USA) or myristyl alcohol (Speziol®C14 Pharma, available from Cognis, Germany) are used.

Co-solvents that may be used, include, but are not limited to, polyethylene glycols, glycerin, polyethylene glycols of various molecular weights and the like and mixtures thereof, propylene glycol, caffeine, xanthenes, gentisic acid, isopropyl alcohol, transcutol, vegetable oil, silicon oil, lanolin, refined animal oil, isopropyl myristate, jojoba oil, almond oil, avocado oil, coconut oil, capric-caprylic triglyceride of fractionated coconut oil, nutmeg oil, PEG-6 apricot kernel oil (e.g., oleyl polyoxylglycerides: Labrafil®M 1944 CS), castor oil, olive oil and oleic acid, soybean oil, sunflower oil, peanut oil, canola oil and the like and mixtures thereof. Preferably, the co-solvent is polyethylene glycols.

The emulsifier in the composition of present invention is generally a nonionic, anionic, cationic or amphoteric surfactant. Suitable emulsifiers include, but are not limited to, glyceryl stearate, polyethylene glycol 100 stearate, glyceryl monostearate, polysorbate 60, sorbitan monostearate, polyglyceryl-4 oleate, polyoxyethylene(4)lauryl ether or trivalent cationic and the like, and sodium lauryl sulphate and mixtures thereof. Preferably, glyceryl stearate.

The phrase “chelating agent”, as used herein, is intended to mean a compound which complexes with the metal ions in the formulation and possibly alleviates the occurrence of unwanted chemical reactions. Such compounds include, by way of example and without limitation, ethylene diamine tetraacetic acid (EDTA), and the like.

The phrase “buffering agent”, as used herein, in the composition of present invention, is selected from the group comprising disodium phosphate dihydrate, potassium metaphosphate, potassium phosphate, monobasic sodium acetate and sodium citrate. The preferred buffering agent is disodium phosphate dihydrate.

The composition of the present invention optionally contains preservatives such as methylparaben, propylparaben and benzyl alcohol. The preferred preservative is benzyl alcohol.

Anti-oxidants, which can be used in the composition of the present invention, are selected from butylatedhydroxytoluene, and mixtures thereof. Butylatedhydroxytoluene (BHT) is the preferred anti-oxidant.

Generally, the percentages of the individual components of the pharmaceutical composition of the present invention are set forth in Table 1.

TABLE 1

| Sr. No. | Ingredient | % w/w |
|---|---|---|
| 1. | Betamethasone Dipropionate | 0.0643 |
| 2. | Calcipotriene anhydrous | 0.005 |
| 3. | Super Refined Oleyl Alcohol (Super Refined | 1.0-15.0 |
| | Novol ® NF) or Myristyl alcohol (Speziol ®C14 | |
| | Pharma, available from Cognis, Germany) | |
| 4. | Butylated Hydroxy Toulene | 0.1-4.0 |
| 5. | Mineral oil (Drakeol ® 35) | 1.0-10 |
| 6. | White petrolatum (Ultima ®) | q.s. to 100 |
| | Total weight | 100 |

The following examples are provided to enable one skilled in the art to practice the invention and are merely illustrative of the invention. The examples should not be read as limiting the scope of the invention as defined in the claims.

### EXAMPLES:

### Example 1

### Preparation of Topically Applicable Ointment Composition of Calcipotriene 0.005% and Betamethasone Dipropionate 0.0643%.

The ingredients and amounts for this example are set forth below in Table 2.

TABLE 2

| Sr. No. | Ingredient | % w/w |
|---|---|---|
| 1. | Betamethasone Dipropionate | 0.0643 |
| 2. | Calcipotriene anhydrous | 0.005 |
| 3. | Super Refined Oleyl Alcohol (Super Refined | 10.0 |
| | Novol ®NF) | |
| 4. | Butylated Hydroxy Toulene | 2.0 |
| 5. | Mineral oil (Drakeol 35) | 3.0 |
| 6. | White petrolatum (Ultima) | q.s. to 100 |
| | Total weight | 100 |

Manufacturing Procedure:

### 1. Preparation of Ointment Base:

White petrolatum, mineral oil and butylated hydroxytoluene (half part) were heated to about 65° C. to about 70° C. to completely melt.

### 2. Preparation of Drug Solution:

Super refined Oleyl alcohol and butylated hydroxytoluene (remaining half part) were dissolved under stifling and by heating to about 60° C. to about 65° C., then calcipotriene and betamethasone dipropionate are completely dissolved under homogenization.

### 3. Homogenization

Drug solution was added to the ointment base at about 65° C. to about 70° C. under stifling for about 10 minutes, subsequently homogenization carried out for about 30 minutes to about 60 minutes and then cooled to about room temperature under stirring.

### Example 2

### Preparation of Topically Applicable Ointment Composition of Calcipotriene 0.005% and Betamethasone Dipropionate 0.0643%.

The ingredients and amounts for this example are set forth below in Table 3.

TABLE 3

| Sr. No. | Ingredient | % w/w |
|---|---|---|
| 1. | Betamethasone Dipropionate | 0.0643 |
| 2. | Calcipotriene anhydrous | 0.005 |
| 3. | Myristyl alcohol (Speziol ®C14 Pharma, | 3.0 |
| | available from Cognis, Germany) | |
| 4. | Butylated HydroxyToluene | 1.8 |
| 5. | Mineral oil (Drakeol ® 35) | 3.0 |
| 6. | White petrolatum (Ultima ®) | q.s. to 100 |
| | Total weight | 100 |

Manufacturing Procedure:

### 1. Preparation of Ointment Base:

White petrolatum, mineral oil and butylated hydroxy toluene (half part) were heated to about 65° C. to about 70° C. to completely melt.

### 2. Preparation of Drug Solution:

Myristyl alcohol and butylated hydroxy toluene (remaining half part) were dissolved under stirring and by heating to about 50° C. to about 55° C., and then Calcipotriene and Betamethasone Dipropionate are completely dissolved under homogenization.

### 3. Homogenization

Drug solution was added to the ointment base at about 65° C. to about 70° C. under stifling for about 10 minutes, subsequently homogenization carried out for about 30 minutes to about 60 minutes and then cooled to about room temperature under stirring.

Stability data of the combination compositions of Example 2 are described below.

The reference standard was TACLONEX® ointment (a commercial product containing calcipotriene and betamethasone dipropionate, stabilized with a poly (alkylene oxide) ester solvent), which is known to be a stable product. The compositions of Example 2 and TACLONEX® ointment were stored at about 40° C. for 3 months, and their stability with respect to the related substances, was determined after an interval of one month, till the end of the three months. The stability data of betamethasone dipropionate and calcipotriene is provided in Tables No. 4 and 5 respectively.

Based on the results provided in Tables 4 and 5, the compositions of the present invention prepared with myristyl alcohol, were found to be stable in an accelerated stability condition. Note the assay values of Example 2 composition of the present invention is comparable to the control (Taclonex®)

TABLE 4
Betamethasone Dipropionate

| Time period/ | | | | | 17-propionate, | |
|---|---|---|---|---|---|---|
| condition | Product | Assay | 17-propionate | 21-propionate | 21 acetate | 6 Bromo |
| Initial | Taclonex | 99.7 | 0.04 | 0.05 | 0.04 | 0.03 |
| | Example 2 | 100.1 | 0.08 | 0.03 | ND | 0.03 |
| 1M/40° C. | Taclonex | 99.4 | 0.15 | 0.19 | 0.04 | 0.07 |
| | Example 2 | 101.1 | 0.20 | 0.04 | 0.03 | 0.04 |
| 2M/40° C. | Taclonex | 99.9 | 0.16 | 0.12 | 0.05 | 0.05 |
| | Example 2 | 100.5 | 0.17 | 0.03 | 0.03 | 0.03 |
| 3M/40° C. | Taclonex | 99.6 | 0.05 | 0.06 | 0.03 | 0.05 |
| | Example 2 | 100.0 | 0.12 | 0.03 | 0.03 | 0.03 |

TABLE 5
Calcipotriene

| | | | | | | Unknown | Unknown | |
|---|---|---|---|---|---|---|---|---|
| Time period/ | | | | | | At | At | Total |
| condition | Product | Assay | Imp-B | Imp-C | Imp-D | RRT1.62 | RRT1.64 | impurity |
| Initial | Taclonex | 99.3 | ND | 0.61 | 0.51 | ND | ND | 1.14 |
| | Example 2 | 97.1 | ND | 0.21 | 0.19 | 0.09 | 0.08 | 0.75 |
| 1M/40° C. | Taclonex | 99.9 | ND | 0.68 | 0.70 | ND | ND | 2.44 |
| | Example 2 | 98.1 | ND | 0.14 | 0.25 | 0.08 | 0.15 | 1.03 |
| 2M/40° C. | Taclonex | 99.0 | ND | 0.66 | 0.65 | ND | ND | 2.70 |
| | Example 2 | 95.0 | ND | 0.14 | 0.18 | 0.04 | 0.09 | 0.84 |
| 3M/40° C. | Taclonex | 98.9 | ND | 0.54 | 0.66 | ND | ND | 2.43 |
| | Example 2 | 96.6 | ND | 0.14 | 0.18 | 0.10 | 0.06 | 0.95 |

Wherein, (Impurity=IMP)>IMP B, IMP C & IMP D are chemically defined as
IMP B-(5Z, 7Z, 22E, 24S)-24-cyclopropyl-9, 10-secochola-5, 7, 10(19), 22-tetraene-1α, 3β, 24-triol
IMP C-(1α,3β,,5E,7E,22E,24S)-24-cyclopropyl-9,10-secochola-5,7,10(19),22-tetraene-1,3,24-triol
IMP D-(1α,3β,5Z,7E,22E,24R)-24-cyclopropy -9,10-secochola-5,7,10(19),22-tetraene-1,3,24-triol

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention.

1. A stable topical composition comprising: (a) a vitamin D or vitamin D analogues (b) a corticosteroid; and (c) a straight chain C₆-C₂₈ fatty alcohol.

2. The composition of claim 1, wherein the vitamin D or vitamin D analogue compound is anhydrous calcipotriene.

3. The composition of claim 1, wherein the corticosteroid is betamethasone or its pharmaceutically acceptable salts.

4. The composition of claim 1, wherein the straight chain C₆-C₂₈ fatty alcohol is either oleyl alcohol or myristyl alcohol or mixtures thereof.

5. A stable ointment composition suitable for the treatment of psoriasis comprising: (a) a vitamin D or vitamin D analogues (b) a corticosteroid; and (c) a straight chain C₆-C₂₈ fatty alcohol in an ointment base containing petrolatum and/or mineral oil, optionally containing an antioxidant.

6. The composition of claim 1 wherein the antioxidant is butylatedhydroxytoluene.

7. A stable ointment composition comprising 0.005% w/w of calcipotriene anhydrous, 0.0643% w/w of betamethasone dipropionate, 1.0-15.0% w/w of oleyl alcohol or myristyl alcohol, 0.1-4.0% w/w of butylatedhydroxytoluene, 1.0-10.0% w/w of mineral oil and 2.0-98% w/w of white petrolatum.
